(19) United States
(12) Patent Application Publication
(10) Pub. No.: US 2008/0027431 A1
(43) Pub. Date: Jan. 31, 2008

(54) Coated medical device and method of making

(76) Inventors: James M. Williams, Knoxville, TN (US); C. Christopher Klepper, Radford, VA (US)

Correspondence Address: ROBERT J. LAUF, 998 W. OUTER DRIVE, OAK RIDGE, TN, 37830, US

(21) Appl. No.: 11/880,584

(22) Filed: Jul. 23, 2007

Related U.S. Application Data
(60) Provisional application No. 60/833,933, filed on Jul. 29, 2006.

(51) Int. Cl. A61B 17/68; A61B 17/32

(52) U.S. Cl. 606/60; 164/113; 164/75; 420/417; 420/422; 420/427; 420/435; 420/501; 420/507; 420/8; 427/576; 606/167

(57) ABSTRACT

A boron coating is applied to selected surfaces of surgical devices, including orthopedic prosthetic alloys and surgical tools to improve sliding wear and provide other benefits. The coating may be applied to CoCrMo, stainless steel, or titanium alloy components of artificial hips, knees, shoulders, and other articulating joints, which bear on wear partners and which experience rubbing wear during use of the joint. In such cases, the coating is preferably applied in particular to the rubbing wear surface of the CoCrMo alloy component to reduce wear of either the alloy component or the mating polymer component, for corrosion improvement, for biocompatibility, or for improvement in infection performance of the device. The boron coating may show evidence for reactive bonding to the substrate, which may include evidence of in-diffusion or of interdiffusion between the boron and the substrate material. The inventive boron or boron-based coating may also be applied to non-wear surfaces of the alloy to improve performance in terms of biocompatibility or infection resistance, as for hip joint stems or other fixation parts of devices. The inventive coating may also be applied to medical devices such as surgical tools to provide a hard, sharp cutting surface. A method of making boron-coated medical devices is also disclosed.

### CROSS-REFERENCE TO RELATED APPLICATION

This application claims the benefit of Provisional Patent Application No. 60/833,933 filed on Jul. 29, 2006 by the present inventors, the entire disclosure of which is incorporated herein by reference.

### STATEMENT REGARDING FEDERALLY SPONSORED RESEARCH

This invention was made with Government support under Contract No. 1R43-AR05126201 awarded by the U.S. National Institutes of Health to HY-Tech Research Corp. and the Government has certain rights in this invention.

### BACKGROUND OF THE INVENTION

1. Field of the Invention

The invention pertains to the field of medical devices and more particularly to medical devices having articulating surfaces such as joint prostheses and the like.

2. Description of Related Art

Well over 90% of the total joint arthroplasties performed are classified as “successful” at present, meaning that they last approximately as long as expected or until the patient dies from other causes. Success, however, depends in part on patient selection with respect to life expectancy, weight, activity expected and other factors. It does not mean that the devices last forever. It is accepted that a large fraction of failures, problems, and limitations of the practice are caused by osteolysis (bone loss) where the osteolysis is promoted by tissue reaction to particulate wear debris from the polymer component of these total joint systems, which is typically ultrahigh molecular weight polyethylene, UHMWPE. (At the juncture of articulation, artificial joints typically comprise a polished alloy bearing surface working against a mating load bearing surface of UHMWPE, where the latter is the surface of a compliant “pad” of some thickness.) Bone loss ultimately leads to loosening of the respective joint components in their fixations. It is believed that certain particle size fractions of polymer wear debris are immunogenic and can lead to inflammation. In addition, it is thought that the polymer debris can also promote infection, which can also cause failure of the system (septic failure). Reduction of UHMWPE wear debris would therefore improve the performance of the devices.

At present, the most commonly used metal component in these devices is made from a CoCrMo alloy. It is postulated that wear of the UHMWPE component rubbing against the alloy surface may be caused by irregularities or asperities on the polished surface of the alloy, which cannot be removed by polishing. In the best polishing limit, the protruding asperities consist of the well-known precipitates of a carbide phase that provide hardening for the alloy. These carbides cannot be polished down because the difference in hardness between the carbide and the alloy matrix inherently creates surface relief. The extent of the carbide protrusion is of the order of 10 to 30 nm. The average surface roughness remaining after polishing appears to be limited by this intrinsic factor, given that the respective plateaus of the carbides and the valleys may themselves have a roughness of less than 1 nm. In addition, the demarcation between the carbides and the softer matrix is rather sharp, rendering the carbide phases as “mesa-like” with rapidly rising edges. It is suspected that the mechanism of UHMWPE wear could involve this intrinsic polishing topography, possibly through snagging of polymer chains by the protruding carbide phases. The hardness of the carbides is a factor of three greater than for the matrix.

Alternatively, titanium alloys, where Ti—6Al—4V is the most popular, have been used in the past, but these alloys have other wear issues that are more severe than for the CoCrMo alloy. The polishing limitation mentioned for the CoCrMo alloy does not apply to Ti—6Al—4V alloy, however, as that alloy can be polished to a virtually optic smoothness of less than 1 nm RMS. The passive oxide on the alloy chemically screens against ion release very well if undisturbed. This property accounts for the fact that the biocompatibility has been regarded as perhaps the best of any material if the oxide is undisturbed. Unfortunately, the oxide is friable and is easily removed from the surface by rubbing. The amorphous oxide is also putatively harder than the alloy. Under hard rubbing by the UHMWPE component, the oxide comes off in the interface and some becomes embedded in the UHMWPE component. The system takes on the quality of three-body wear with the oxide particles acting as the abrasive. Wear then becomes autocatalytic as the more wear debris that is generated, the more acute the surface abrasion is, and further oxide is generated as the alloy continually attempts to re-passivate its surface. Surrounding tissue becomes blackened by oxide wear debris and amalgam of UHMWPE with oxide wear debris. Thus, although the wear mechanism is completely different from that of the CoCrMo alloy, the history of wear performance for Ti—6Al—4V is so poor that the alloy has fallen almost totally out of favor for use where wear is a factor. Note that individual processes such as ion implantation or surface treatments are not approved per se by the FDA. Only devices with their total content and specified manufacturing steps are approved.

There are hip joint systems with Ti alloy stems for which the heads may be of CoCrMo or ceramic material. This creates yet another need, viz., some patients are acutely sensitive to one of the metals in CoCrMo - probably Cr. While the option of a Ti alloy stem and a ceramic head might be available for hip joints, there are fewer options for hybridization of knee joints. Therefore, a solution to the surface wear issues might ultimately create a pathway toward wider use for Ti alloys in prosthetic devices.

One approach to hardening prosthetic devices consists of implanting the surface with high energy nitrogen (N) ions. As originally manifested, the process was for treatment of Ti—6Al—4V alloy, for which wear was severe. U.S. Pat. No. 7,182,784 discloses execution of the process at the commercial level without discoloration of the product. The key amounted to good vacuum practice. Later, publications demonstrated advantages for virtually the same ion implantation treatment for the widely used CoCrMo alloy. That practice persists at the commercial level; however, Applicants are not aware of any patents on the subject. Ion implantation practice illustrates a key point about use of coatings in orthopedic hard-rubbing wear situations, however. They absolutely must not come off. That is one reason why the ion implantation process was so readily accepted. High-energy introduction of the N atoms into the substrate one atom at a time guarantees no delamination.

U.S. Pat. Nos. 5,192,323 and 5,326,362 disclose high temperature surface nitridation of titanium orthopedic implant devices by exposure to molecular nitrogen at temperatures from 750 to 1300° C. U.S. Pat. No. 5,308,412 discloses a similar treatment for CoCr or CoCrMo alloys.

U.S. Pat. No. 5,370,696 discloses a plasma coating process for depositing a polymer coating onto a prosthetic implant, wherein polymer particles are passed through a plasma discharge and the molten particles are deposited over the implant surface.

U.S. Pat. No. 5,372,660 discloses surface oxidation treatment of titanium alloys containing zirconium to form a hard, adherent oxide layer.

U.S. Pat. No. 5,380,547 discloses plasma-activated nitridation of orthopedic implant devices.

U.S. Pat. No. 5,498,302 discloses surface-hardening of orthopedic alloys by more-or-less conventional methods of adding hardening agents such as N, C, or O. However, special reactive alloy constituents are added to the alloys to form stronger or harder precipitates than might otherwise exist with the basic substrate elemental components. The purpose is to prevent wear of the alloy, not the UHMWPE.

U.S. Pat. Nos. 5,702,448 and 5,868,796 disclose the application of TiN or ZrN coatings by plasma deposition to portions of orthopedic prosthetics that may be in contact with bone, presumably in instances where bone in-growth is the method of fixation, rather than cement. They further disclose the application of similar coatings to wear surfaces of implants.

U.S. Pat. No. 5,704,239 discloses shot-peening of the surface of titanium-based orthopedic implants to improve fretting wear and create a compressively stressed surface region.

U.S. Pat. Nos. 6,585,772, 6,726,725, 7,070,623, and 7,182,784 disclose devices and methods involving surface oxidation of zirconium or zirconium-based alloys. The treatment consists of diffusion in to and oxidation of the surface by oxygen. It is a high temperature treatment. The treatment is claimed not to be a coating because of the chemically reactive nature of the process, based as it is on the high chemical free energy of formation of zirconium oxide (zirconia). The surface amounts to a hard ceramic. The process is claimed to produce advantages in wear of the UHMWPE over standard non-coated CoCrMo. Because of the reactive nature of the formation process, the process is claimed not to be a coating and therefore it cannot come off. For the Zr case that appears to be true. However, it should be noted that having a reactive process is probably a necessary but not sufficient condition to guarantee adhesion. For the titanium-oxygen system, similar in chemical principles to the zirconium-oxygen system, the oxide spalls off and is a huge problem. A principal advantage for the treatment is the depth of the ceramic of 5 μm.

### Objects and Advantages

Objects of the present invention include the following: providing medical devices having improved wear properties; providing articulating prostheses having improved wear properties; providing articulating prostheses capable of improved surface polishing and/or having inherently smoother wear surfaces; providing medical devices capable of maintaining a sharp edge; providing articulating prostheses having less tendency to produce wear debris; providing articulating prostheses having lower immunogenic tendencies; providing a method for making articulating prostheses having improved wear properties; providing a method for making alloy components for articulating prostheses capable of accepting and retaining a superior surface polish; and, providing a method of making articulating prostheses having improved chemical stability and improved biocompatibility. These and other objects and advantages of the invention will become apparent from consideration of the following specification, read in conjunction with the drawings.

### SUMMARY OF THE INVENTION

According to one aspect of the invention, a medical device comprises: a metal component; and, an adherent coating comprising elemental boron deposited on a selected surface of the metal component.

According to another aspect of the invention, method for making a medical device comprises the following steps: (a) forming a metal component; and, (b) depositing an adherent coating comprising elemental boron on a selected surface of the metal component.

According to another aspect of the invention, an implantable prosthetic device comprises: a metal component having an articulating surface configured to engage a mating component, the mating component comprising a polymer; and, an adherent coating comprising elemental boron deposited on the articulating surface of the metal component, whereby the coating may engage the mating component throughout the useful range of articulation of the prosthetic device.

### BRIEF DESCRIPTION OF THE DRAWINGS

The drawings accompanying and forming part of this specification are included to depict certain aspects of the invention. A clearer conception of the invention, and of the components and operation of systems provided with the invention, will become more readily apparent by referring to the exemplary, and therefore non-limiting embodiments illustrated in the drawing figures, wherein like numerals (if they occur in more than one view) designate the same elements. The features in the drawings are not necessarily drawn to scale.

FIG. 1 is a schematic diagram of a plasma source useful in carrying out the present invention.

FIG. 2 is a schematic diagram of two types of particulate filters for use in a plasma deposition system.

FIG. 3 is a comparison of wear tracks in coated and uncoated samples exposed to sliding wear.

FIG. 4 shows data from Rutherford backscattering spectroscopy measurements on a sample of CoCrMo alloy coated with B according to the present invention.

### DETAILED DESCRIPTION OF THE INVENTION

Based on the aforementioned needs identified in considering existing methods, Applicants postulated that the effect of alloy carbides in producing wear of the mating UHMWPE component might be reduced by the addition of a coating, which might provide some “buffering,” to reduce the sharpness of the up-thrust of these carbides, while also, perhaps, providing some other favorable properties. Applicants determined that elemental boron, has several attractive properties as a candidate for such a coating:

First, an atomically deposited smooth hard-coating whose thickness is several times the dimension of the carbide up-thrust could help buffer the sharpness or inherent abrasiveness of the carbide edges in the residual carbide polishing topography, and thus reduce the tendency for snagging of polymer chains at the nanometer level by the CoCrMo alloy.

Second, finding a suitably adherent coating for CoCrMo and other such alloys is difficult because their passive oxides provide a barrier to adhesion, and these oxides are quite stable and reform quickly even during vacuum coating processes.

Third, elemental boron is 3-valent and thus has a strong tendency to react with other elements including those of transition metals, of which these alloys are composed. In contrast, hard ceramic compounds such as SiC or TiN have closed pair bonding due to the covalent nature of the compound itself, and have little chemical potential left over for reactive bonding with substrates. The same applies to elemental carbon (e.g., “diamond-like carbon”). The superiority of elemental boron is supported by thermodynamic calculations, which suggest the possibility of strong reactive bonding of boron to transition metals comprising the subject alloys.

Moreover, the inherently high adhesion of elemental B to the substrate is further aided by the relatively high energy of arrival (up to 600 eV) of the coating atoms when using the cathodic arc technique according to another aspect of the present invention. This energy helps the B to penetrate the oxide and actually causes some sputtering of the oxide. The process is also aided by the rapid deposition intrinsic in the cathodic arc process, relative to the rate of returning contaminant atoms from the residual vacuum.

Fourth, in its common forms of the icosahedral structure or amorphous structure, elemental boron is the hardest element other than diamond. The hardness value of 33 GPa, is higher than the hardness value of SiC, TiN, and in fact, most other industrial hard compounds. This value may be compromised slightly by deliberate additions of other chemical species according to other aspects of the invention to be described below.

Fifth, the nature of the passive oxide on boron is a key factor in performance, as is the case with the oxides of alloy substrates themselves (without coatings). The passive oxide prevents ion release in aqueous saline or acidic solutions, particularly ones based on halides. Boron is corrosion resistant even in boiling hydrofluoric acid (HF). As a result, boron is expected to be biocompatible. Furthermore, under wear, any oxide removed may exist as the hydrated oxide, which is boric acid (or eyewash). This product could act as a mild antiseptic and is also claimed to provide exceptional lubricity in itself [see, for example, A. Erdemir et al., “A study of the formation and self lubrication mechanisms of boric acid films on boric oxide coatings,” Surface and Coatings Technology, 43/44, 588-596 (1990)].

In one aspect, the invention comprises a boron coating for surgical devices and orthopedic prosthetic alloys. In one preferred embodiment, the coating may be applied to CoCrMo alloy components of artificial hips, knees, shoulders, and other articulating joints, which bear on wear partners and which experience rubbing wear during use of the joint. In this case, the coating is preferably applied in particular to the rubbing wear surface of the CoCrMo alloy component to reduce wear of either the alloy component or the mating polymer component, for corrosion improvement, for biocompatibility, or for improvement in infection performance of the device. The boron coating as described above may show evidence for reactive bonding to the substrate, which may include evidence of in-diffusion or of inter-diffusion between the boron and the substrate material. The inventive boron or boron-based coating may also be applied to non-wear surfaces of the alloy to improve performance in terms of biocompatibility or infection resistance, as for hip joint stems or other fixation parts of devices.

One preferred means for depositing a boron or boron-based coating onto surgical and orthopedic alloy components comprises generating a boron plasma from a solid boron cathode in a cathodic arc ion source (also referred to as a vacuum arc ion source) and then providing for streaming of this plasma onto a selected surface of the alloy component. The streaming technique may further involve filtering of macroscopic particles from the stream, manipulation and electrical polarization of the component to improve coating of surface contours, and electrical polarization to add further energy to the incoming ions for improved properties and adhesion.

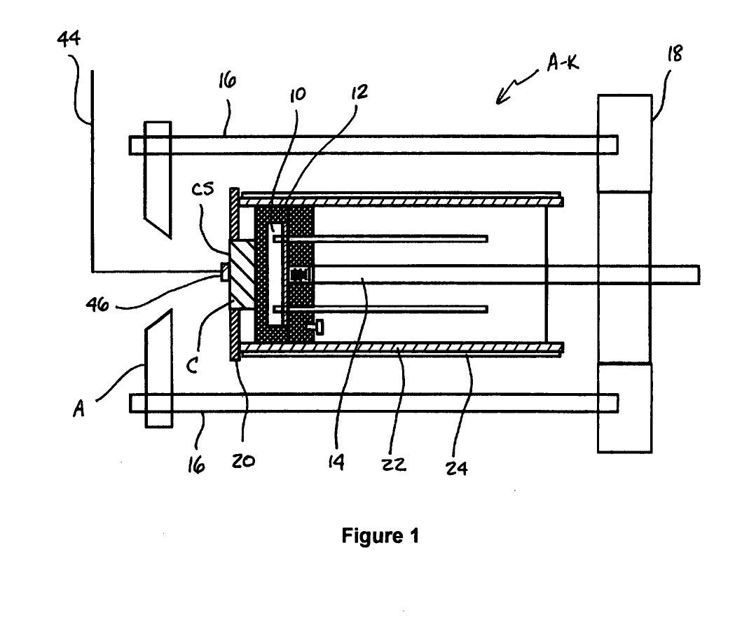

The cathodic arc process used herein is that generally taught by Klepper et al. in U.S. Pat. No. 6,495,002, the entire disclosure of which is incorporated herein by reference. The sintered, pure boron cathode was prepared as generally taught by Morrow et al. in U.S. Pat. No. 6,562,418, the entire disclosure of which is incorporated herein by reference. FIG. 1 shows the general construction of the deposition system. The anode cathode configuration is classic with a disk shaped cathode, preferably 25 mm in diameter, and a ring anode of a comparable inner diameter, and preferably positioned at about 25 mm from the cathode. The anode was made by e-beam machining of a cast tungsten disk.

The anode-cathode assembly A-K as taught by Klepper et al. '002 is illustrated in FIG. 1. A conventional resistive heater 10 is enclosed inside a housing 12, preferably formed of graphite. A consolidated cathode C, produced with microwave energy in a frequency range of about 2-30 GHz, is firmly attached to the housing 12 with a high temperature and vacuum compatible paste. Preferably, cathode C is formed of pure boron. The arc current is conducted to the cathode C via a conventional electrical vacuum feed-through 14. The anode A, on the other hand, is electrically connected to the ground-side of the arc power supply. In the present work, the source was modified so that current is conducted to the anode via a water-cooled Cu—W alloy manifold. The anode A is preferably made of tungsten, a metal compatible with high-temperatures, which allows for operation without active cooling of this component for up to about 1 second.

The cathode C is preheated to a temperature of about 800° C. and more preferably about 800-1300° C., by heater 10.

An insulating ceramic shield 20, preferably formed of boron nitride (BN), surrounds the cathode C, for limiting the footprint of the discharge to the cathode surface CS, and, contributing to a more directed flow of the ionized vapor toward the deposition region of the vacuum chamber. In FIG. 1 reference numerals 22 and 24 designate heat shields, preferably formed of alumina ceramic and tantalum materials respectively.

For triggering of the arc, preferably a molybdenum trigger wire 44 is connected to the front of the cathode surface CS, via an insulating ceramic block 46. The ceramic block is preferably coated with a carbonaceous substance. A high voltage electrical pulse between the wire 44 and the cathode C produces an electrical breakdown across the ceramic surface, releasing an ionized vapor that fills the A-K gap and allows for the startup up of the vacuum arc discharge. This triggering process is akin to similar techniques known in vacuum arc technology “surface discharge” or “surface breakdown” triggering.

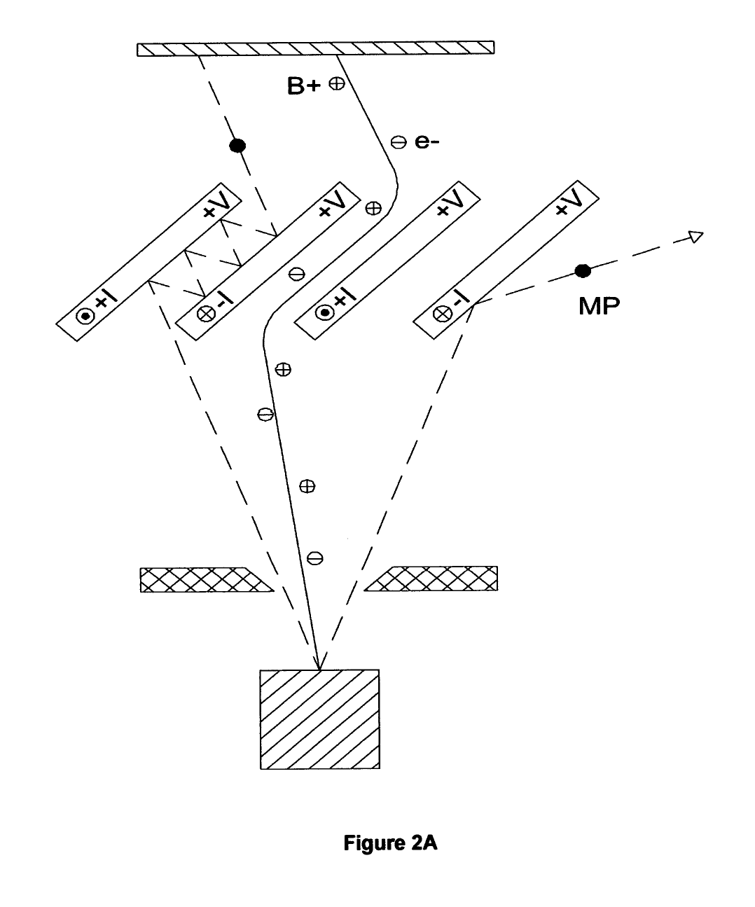

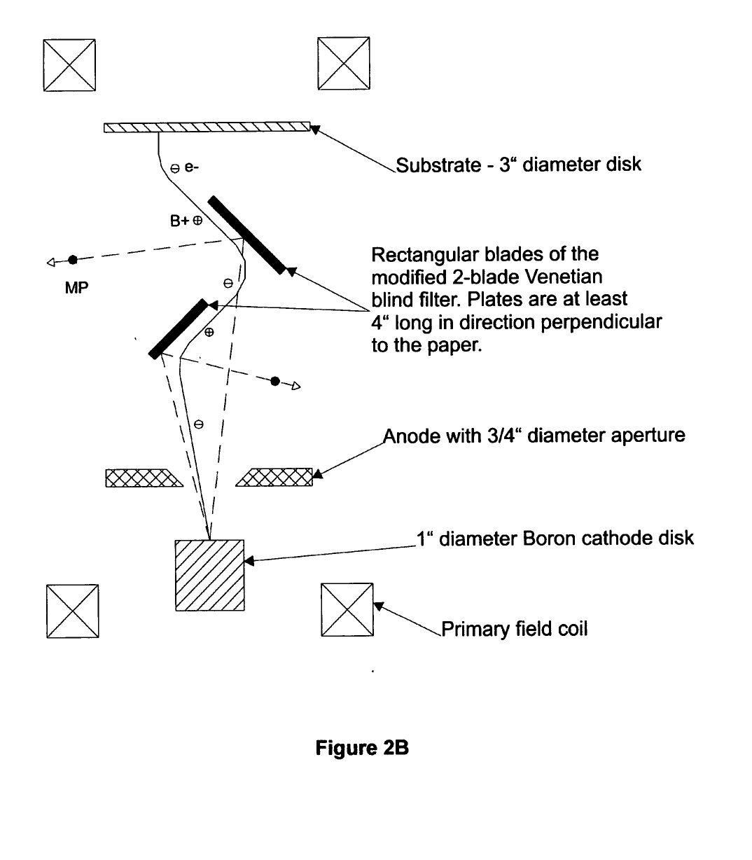

It is desirable to reduce or eliminate the ability of macroscopic particles occasionally created at the target from reaching the substrate, because these particulates may create defects in the coating. One approach is the so-called Venetian blind filter as shown schematically in FIG. 2. The design shown in FIG. 2A uses parallel plates with current (˜200 A) flowing alternatively into or out of the plane of the drawing. Metal macroparticles (usually liquid) will likely stick to the plates or quickly lose energy. Macroscopic particles of boron, on the other hand, are nearly always solid and highly elastic, and to improve elimination of these particles, Applicants modified the filter design as shown generally in FIG. 2B. The approach is to draw enough current in the plates to perturb the main field and guide the boron plasma through the filter. For the macroparticles, there is no possible direct path to the substrate.

The following examples will illustrate various features of the inventive method and its application to material systems of interest for medical devices.

### EXAMPLE

Flat coupons of Co—Cr—Mo alloy, pre-polished to the orthopedic industry polishing standards for test samples, were coated with boron to thicknesses varying from about 150 nm to as much as 400 nm, in regions clearly distinguishable by the color of the coating. Thinner coatings have a light blue color, whereas thicker coatings exhibit a tan color; beyond 500 nm, amorphous boron is essentially grey; crystalline boron would be black. It is noted that even the thin (150 nm) coating amounts to several times the thickness of the typical carbide protrusion. The coatings were adherent and substantially continuous over selected areas of the substrate.

Deposition parameters were as follows:
Cathode: Sintered boron, heated to ˜1000° C.
Substrate: Cast F5 alloy, polished to less than 3 μ-inch or ˜75 nm
Substrate bias: −500V with respect to chamber ground and with spark suppression
Substrate temperature: About 100° C. initially, increasing to about 150° C. during the deposition
Base pressure: About 5×10⁻⁷ Torr (˜10⁻⁴ Pa) with the cathode hot
Deposition rate: Typically ˜2-15 nm/s (with particle filter)

The modified filter shown in FIG. 2B reduced particulate incorporation to less than 2 particles per cm² per 100 nm of deposition. This made it possible to find regions totally devoid of particles for the wear test.

### EXAMPLE

A setup designed for reciprocating pin-on-disk motion, with the disk (coated sample) moving, while the pin is stationary, was modified to include a reservoir around the sample allowing the sample to be submerged in a saline solution. The setup allowed for selectable static loads to be placed on the pin. The specially made UHMWPE pins were 6 mm diameter rods with hemispherically formed contact ends. The load was 10 N. The traverse was purely linear and cyclic with a 1 s period and a stroke of 6.7 mm. The test duration was 24 h. The immersion medium was saline at 3.5 wt. % NaCl in distilled H₂O. The combined elasto/plastic properties of the pin were such that the contact area under the 10 N load was approximately 0.8 to 1.0 mm in diameter.

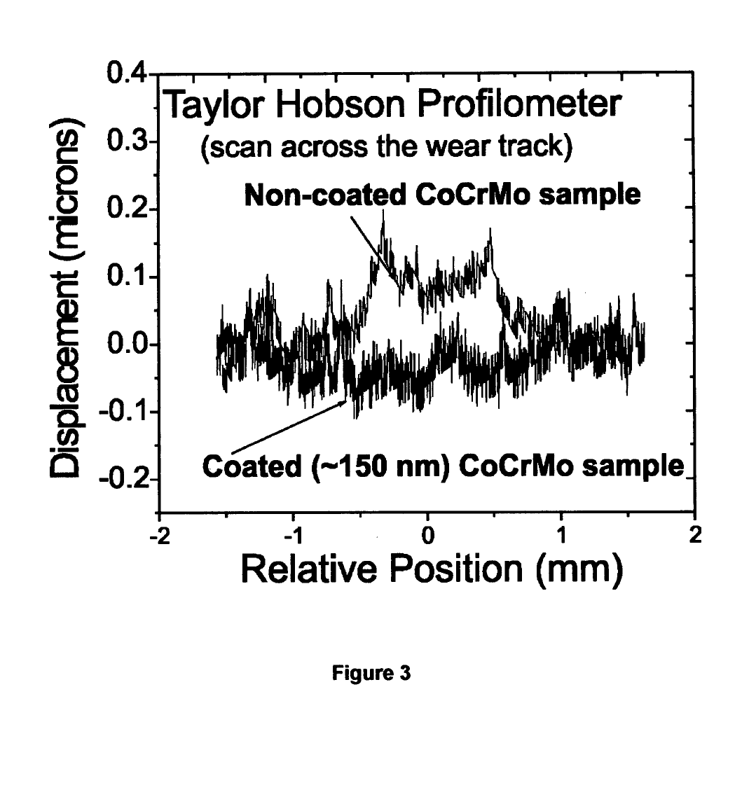

The results of these wear tests were as follows. In comparison with uncoated Co—Cr—Mo, the friction coefficient was reduced by approximately a factor of two for the boron-coated coupon. That means from approximately 0.02 for the uncoated to approximately 0.01 for the coated. Thus, friction was not a large issue in either case, and the coefficient varied somewhat over the course of the test for both cases. FIG. 3 shows profilometry traces across the wear track on the Co—Cr—Mo disk after a 24 h run. These reveal a transfer film of about 150 nm for the uncoated sample and no detectable change in topography for the coated sample. Rutherford backscattering (RBS) analysis of the wear track region of the coated samples confirmed that the thickness of the boron coating remained unchanged; i.e. there is no loss in coating material.

### EXAMPLE

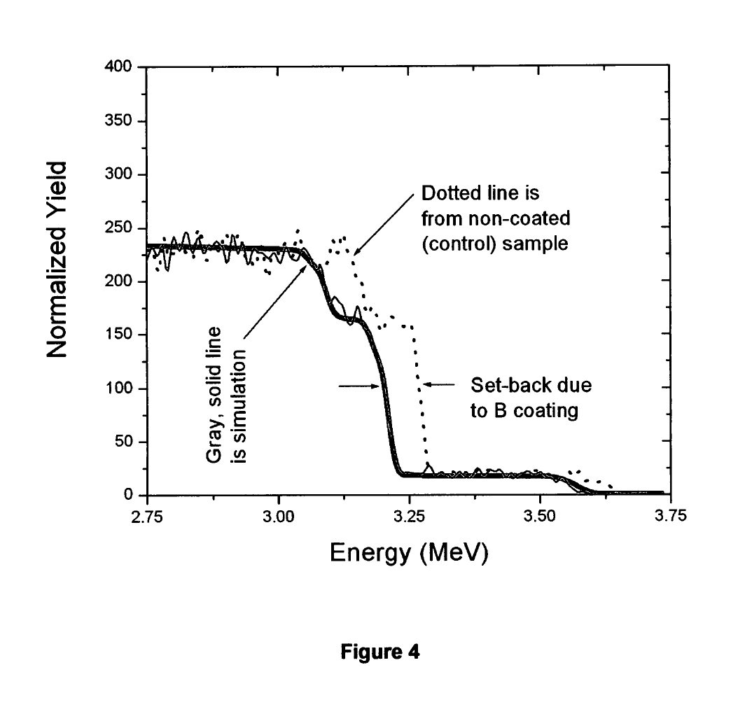

One coating sample was estimated to be about 150 nm thick based on RBS analysis. That was the thickness intended, and the RBS measurement thus demonstrates that the deposition process is easily controlled to achieve the desired coating thickness. The RBS analyses further contained evidence of interdiffusion of the coating and substrate phases or of in-diffusion of the boron. This result reflects the expected reactivity of boron as mentioned earlier. The result is shown by some broadening of the Co edge for Co under the coating for the coated material (FIG. 4), as compared with the shape of the edge for bare material. Apparently the Co and the Cr are mostly in the matrix phase and the Mo is mostly in the carbide phase. Thus, the broadening for the Co would correspond to interdiffusion in the matrix phase. Auger analysis indicated that most of the Mo was in the carbide phase. For the carbide phase, there was sometimes more dramatic evidence of intermixing. In that case some of the Mo (apparently in the carbide phase) is not covered up at all, and some Mo is still found all the way out to the surface.

### EXAMPLE

Biocompatibility testing was separated into several components including: (1) characterizations of specimen surface microtopographics by optical and scanning electron microscopy; (2) alterations of surface microtopographics and solutions through exposure of disks within phosphate buffered saline (PBS); (3) post exposure disk surface microtopography and elemental chemistries of the solutions (PBS); (4) fibroblast cell culture assessments using an aliquot of solution (PBS) and contact inhibition exposure techniques; and (5) standardized injections of individual types of particulates and mixtures into synovial pouch regions of rats for assessments of cellular (histological) and matrix (cytokine) responses. In each situation, the base alloys of cobalt and titanium were used in bulk and particulate forms as controls (known biomaterials by ASTM FO4 standards). Also, in each situation, a “worst case” scenario was selected to test limits of the systems, e.g. rough as-machined surfaces on the disks and millions of small individual particles and mixtures of particles injected into the synovial pouches.

Overall preliminary conclusions from these tests on as-machined, coated and control disks and particulates, for biocompatibility profiles, showed that the boron additions to the cobalt alloy substrate were similar to the alloy substrates and somewhat less reactive for the thinner coating condition while the injections of particulates alone and in combinations showed anticipated profiles (similar to controls) for these type simulated debris products.

As noted earlier, elemental boron has extremely high hardness and elastic modulus. Those skilled in the art will appreciate that in some circumstances it may be desirable to reduce the elastic modulus and hardness by a controlled amount to achieve better matching to the wear partner, to lower residual stresses, and to provide better stress relief in the presence of stress concentrators. Applicants have determined that elements such as C, O, and N may be introduced into the B coating in quantities preferably less than about 10% and more preferably less than about 5% to adjust its elastic properties. For instance, introducing 5% O into boron reduces its hardness from about 33 GPa to about 27 GPa. Oxygen and nitrogen may be introduced by a controlled leak of gas into the plasma chamber. Carbon may be added to the boron target either as the element or as B₄C.

In summary, based on the foregoing exemplary embodiments of the invention, it will be appreciated that the inventive boron coating with its native oxide may be superior in many instances to passive oxide surfaces of prior surgical alloys in terms of biocompatibility and resistance to infection. In addition to articulating prostheses such as knee, shoulder, elbow, or hip implants, other devices that may benefit from the invention include fixation devices and trauma repair devices such as plates, rods, screws, etc., where such components might be made from various conventional metals including Co alloys, Ti and its alloys, Zr and its alloys, Ta and its alloys, and stainless steels. Other applications may include dental components and restorations comprising gold or silver and alloys thereof, including amalgams.

It will be appreciated by those skilled in the art that the metal component may be fabricated by any convenient process. Familiar processes include casting, forging, machining, sintering and other powder-based processes, welding, diffusion bonding, etc. The metal surface may be prepared for coating by various familiar procedures including cleaning, grinding, shaping, polishing, etching, sputtering, etc. A further advantage of the inventive method is that in some cases the as-deposited coating is sufficiently smooth that it requires no further polishing or other finishing steps for articulating components such as femural heads.

It will be further appreciated that the inventive coating may be usefully employed on other medical devices including various surgical tools such as scalpels, knives, scissors, and other cutting instruments to provide a superior surface for creating and maintaining a stable, sharp edge. In these devices, it may be advantageous to apply the coating to selected surfaces and then follow with a further sharpening step which may include familiar operations such as grinding, honing, polishing, lapping, and the like.

In the examples described above, coating thickness generally ranged from less than 140 nm to more than 500 nm. It will be appreciated, however, that the invention is not limited to any particular thickness value per se. It was found that 150 nm is sufficient to blunt the polishing asperities inherent with CoCrMo substrates and improve the friction and wear on an articulating surface, while creating a coating that is cosmetically acceptable without the need for subsequent polishing. For articulating surfaces, Applicants prefer to apply a coating that is no more than 1000 nm (1 μm) thick in order to keep any residual stresses within an acceptable level. For applications where additional surface finishing steps are desired, such as polishing or sharpening, a thicker coating may be applied, particularly if a substrate is selected that has a coefficient of thermal expansion that is closer to that of boron.

Those skilled in the art will appreciate that the coating apparatus may be configured to treat a single workpiece at a time or to treat a plurality of workpieces in one run. The workpiece(s) may be substantially stationary during the coating process, or various fixtures may be employed to rotate or translate the workpiece(s) as the coating is deposited. The coating apparatus may be configured to operate in a substantially batch mode, or it may be configured for continuous or semi-continuous operation through the use of appropriate air locks and other familiar modifications.

As previously noted, ion implantation (using nitrogen ions) is a well-established method for treating some medical devices, particularly hip prostheses. The method of the present invention may be further modified to combine some features of ion implantation with some features of plasma coating to create yet more useful structures. Such modification may be achieved by first operating the inventive system at high voltage to create an implanted layer (implanted with boron atoms rather than nitrogen atoms) and then operating at a lower voltage to build up a boron-rich coating as described generally in the foregoing examples. The advantages of such a treatment include the following: First, it would be an economical way to increase the total thickness of treated material. Second, it could provide a gradation in hardness that would tend to make the coating more robust and better matched to the substrate. Third, previous experience and some of the data presented herein indicate that the hardness of the substrate can be made more uniform, because introduction of impurities into the carbides softens them somewhat, while at the same time, implantation of the matrix hardens it. More uniform hardness could help reduce stress raisers associated with an externally applied stress, because the hard carbides would be less likely to serve as hard “fulcrums” around which the coating is bent as it is pressed into the softer matrix by external loads.

As used herein, terms such as “medical device”, “joint”, “prosthesis”, “arthroplasty”, etc., are intended to include not only devices and procedures for treating humans but also analogous veterinary devices and procedures. In the case of veterinary prostheses, it will be understood that terms such as “hip”, “shoulder”, and “knee” include those joints in humans and the anatomically corresponding joints in animals, even if the latter joints are generally called by other terms in veterinary practice. The term “veterinary” is intended to include the medical treatment of any animal, including companion animals, working animals, livestock, wild animals, circus or performing animals, and captive animals in zoos and like exhibits.

We claim:

1. A medical device comprising:
a metal component; and,
an adherent coating comprising elemental boron deposited on a selected surface of said metal component.

2. The medical device of claim 1 wherein said metal component comprises a metal selected from the group consisting of: Co and its alloys; Ti and its alloys; Zr and its alloys; Ta and its alloys; stainless steels; Au and its alloys and amalgams; and Ag and its alloys and amalgams.

3. The medical device of claim 1 wherein said coating further comprises up to 10% of an element selected from the following group: oxygen; nitrogen; and carbon.

4. The medical device of claim 1 wherein said device comprises an implantable prosthesis having an articulating surface and said coating is applied to at least a portion of said articulating surface.

5. The medical device of claim 1 wherein said device comprises a component selected from the group consisting of: fixation devices; trauma repair devices; plates; rods; and screws.

6. The medical device of claim 1 wherein said device is adapted for cutting tissue and said coating is applied to a selected cutting surface of said device.

7. The medical device of claim 1 further characterized by the interdiffusion of at least one chemical element between said metal component and said coating.

8. A method for making a medical device comprising the following steps:
(a) forming a metal component; and,
(b) depositing an adherent coating comprising elemental boron on a selected surface of said metal component.

9. The method of claim 8 wherein said metal component comprises a metal selected from the group consisting of: Co and its alloys; Ti and its alloys; Zr and its alloys; Ta and its alloys; stainless steels; Au and its alloys and amalgams; and Ag and its alloys and amalgams.

10. The method of claim 8 wherein said metal component is formed by a process selected from the group consisting of: casting; forging; stamping; sintering; and machining.

11. The method of claim 8 wherein said step (b) comprises generating a boron-containing plasma from a solid boron-containing cathode in a cathodic arc ion source and providing for streaming of said boron-containing plasma onto a selected surface of said metal component.

12. The method of claim 11 wherein said step (b) further comprises removing particulate matter from said boron-containing plasma before said plasma contacts said metal component.

13. The method of claim 8 wherein said coating further comprises up to 10% of an element selected from the following group: oxygen; nitrogen; and carbon.

14. The method of claim 8 wherein said device comprises an implantable prosthesis having an articulating surface and said coating is applied to at least a portion of said articulating surface.

15. The method of claim 14 further comprising the following step:
(c) polishing said articulating surface.

16. The method of claim 8 wherein said device comprises a component selected from the group consisting of: fixation devices; trauma repair devices; plates; rods; and screws.

17. The method of claim 8 wherein said device is adapted for cutting tissue and said coating is applied to a selected cutting surface of said device.

18. An implantable prosthetic device comprising:
a metal component having an articulating surface configured to engage a mating component, said mating component comprising a polymer; and,
an adherent coating comprising elemental boron deposited on said articulating surface of said metal component, whereby said coating may engage said mating component throughout the useful range of articulation of said prosthetic device.

19. The implantable prosthetic device of claim 18 wherein:
said metal component is selected from the group consisting of: Co and its alloys; Ti and its alloys; Zr and its alloys; Ta and its alloys; and stainless steels; and,
said mating component comprises ultra high molecular weight polyethylene.

20. The implantable prosthetic device of claim 18 wherein said adherent coating further comprises up to 10% of an element selected from the following group: oxygen; nitrogen; and carbon.
